(19) United States
(12) Patent Application Publication
(10) Pub. No.: US 2007/0275435 A1
(43) Pub. Date: Nov. 29, 2007

(54) CELL CULTURE CHIP AND METHOD FOR REAL-TIME MONITORING OF A CELL CULTURE USING THE SAME

(75) Inventors: Kui-hyun KIM, Yongin-si (KR); Jung-im HAN, Yongin-si (KR); Jun-hong MIN, Yongin-si (KR); Kwang-ho CHEONG, Yongin-si (KR); Byung-chul KIM, Yongin-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/625,847

(22) Filed: Jan. 23, 2007

(30) Foreign Application Priority Data
Jan. 23, 2006 (KR) 10-2006-0006819

(51) Int. Cl. C12Q 1/02; C12M 1/00

(52) U.S. Cl. 435/29; 435/287.1

(57) ABSTRACT

Disclosed herein are a cell culture chip for monitoring a cell culture in real time and a method of monitoring the cell culture using the cell culture chip. The cell culture chip includes a cell culture chamber formed by side walls of a non-conductive material and a bottom layer of an insulating material and capable of accommodating a cell culture media. The cell culture chip also includes a semiconductor layer disposed under the bottom layer, a metal layer disposed under the semiconductor layer, and an electrode disposed in the cell culture chamber. The cell culture chip monitors both the states of cells attached to walls of the cell culture chamber and the states of cells floating in the cell culture media.

This application claims priority to Korean Patent Application No. 10-2006-0006819, filed on Jan. 23, 2006, and all the benefits accruing therefrom under 35 U.S.C. 119, the contents of which are incorporated herein by reference in its entirety.

### BACKGROUND OF THE INVENTION

1. Field of the Invention

The present invention relates generally to a cell culture chip for monitoring a cell culture, and more particularly to a cell culture chip for real-time monitoring of cell cultures in micro scales and a method of monitoring a cell culture using the chip.

2. Description of the Related Art

Cell culture states may be monitored using, for example, cell expension bioreactors equipped with a pH detector. However, although the pH detector can measure the pH of a whole media to expect the cell states, it is unable to detect the states of cells in micro scales that are present in a specific local position.

Japanese Laid-Open Patent Publication No. 2004-113092 describes a cell culture chip made of a transparent plate having a size capable of being placed on a sample die of a microscope and having a well inside, a liquid inlet connected to the well, and a liquid outlet, the chip being characterized as having a sensor for detecting the liquid culture in the well. However, the chip cannot detect the states of cells in micro scales that are present in a specific local position.

Further, International Published Application No. WO 98/054294 describes an apparatus for monitoring cells, including an array of microelectrodes disposed in a cell culture chamber and a standard electrode, each of the microelectrodes having a diameter less than the cell diameter and to which a portion of the cells will be attached, and a method of monitoring cells using the apparatus. However, although the apparatus may be capable of monitoring cells attached to the microarray, it is unable to monitor cells floating in the culture media. Further, it is difficult to manufacture the microelectrodes.

### BRIEF SUMMARY OF THE INVENTION

Aspects of the present invention provide a cell culture chip for real-time monitoring not only the states of cells attached to walls of a culture chamber, but also the states of cells floating in a culture media.

Additional aspects of the present invention also provide a cell culture chip for real-time monitoring not only the states of cells attached to walls of a culture chamber, but also the states of micro-scale cells floating in a culture media.

Further aspects of the present invention also provide a method of real-time monitoring not only the states of cells attached to walls of a culture chamber, but also the states of cells floating in a culture media.

Additional aspects of the present invention also provide a method of real-time monitoring not only the states of cells attached to walls of a culture chamber, but also the states of micro-scale cells floating in a culture media.

In an exemplary embodiment of the present invention, there is provided a cell culture chip for monitoring a cell culture in real time. The cell culture chip includes: a cell culture chamber formed by side walls of a non-conductive material and a bottom layer of an insulating material and capable of accommodating a cell culture media; a semiconductor layer disposed under the bottom layer; a metal layer disposed under the semiconductor layer; and an electrode disposed in the cell culture chamber.

The non-conductive material may be selected from the group consisting of silicone, glass, quartz, and plastics.

The insulating material may be selected from the group consisting of SiO₂, silicone, glass, quartz, and plastics.

The semiconductor layer may be a p-type semiconductor layer.

The metal layer may be made of a material selected from the group consisting of aluminum, platinum, gold, copper, palladium, and titanium.

The electrode may be made of a material selected from the group consisting of platinum, gold, copper, palladium, and titanium.

The metal layer and the electrode may be connected to a measuring means for measuring an electrical parameter.

The electrical parameter may be selected from the group consisting of capacitance, conductance, impedance, resistance, voltage, and current.

A plurality of cell culture chambers may be arranged to form a microarray and each of the cell culture chambers is includes the semiconductor layer, the metal layer, and the electrode.

In a further exemplary embodiment of the present invention, there is provided a method of monitoring cell culture in real time. The method includes: placing a cell culture media and cells to be cultured, into a cell culture chamber of a cell culture chip. The cell culture chip includes the cell culture chamber. The cell culture chamber is formed by side walls of a non-conductive material. The cell culture chip further includes: a bottom layer formed from an insulating material and capable of accommodating the cell culture media; a semiconductor layer disposed under the bottom layer; a metal layer disposed under the semiconductor layer; and an electrode disposed in the cell culture chamber. The method further includes culturing the cells in the cell culture chamber, and measuring an electrical parameter between the metal layer and the electrode.

The culturing of the cells and the measuring of the electrical parameter may be simultaneously performed.

The electrical parameter may be selected from the group consisting of capacitance, conductance, impedance, resistance, voltage, and current.

The method may further comprise converting the measured electrical parameter into a property parameter of the media.

The property parameter may be selected from the group consisting of pH, O₂ concentration, CO₂ concentration, NO concentration, and temperature.

### BRIEF DESCRIPTION OF THE DRAWINGS

The above and other aspects, features, and advantages of the present invention will become more apparent by describing in detail exemplary embodiments thereof with reference to the attached drawings, in which:

FIG. 1 is a side cross-sectional view of a cell culture chip according to an embodiment of the present invention;

FIG. 2A is a photograph illustrating a top of a cell culture chip according to an embodiment of the present invention;

FIG. 2B is an enlarged photograph illustrating rectangular portions of the cell culture chip photograph depicted in FIG. 2A;

FIGS. 3A-3F schematically illustrate a method of preparing a cell culture chip according to an embodiment of the present invention;

FIG. 4 schematically illustrates a process of converting electrical parameters measured between a metal layer and an electrode of a cell culture chip into other electrical parameters, according to an embodiment of the present invention;

FIG. 5 is a graph illustrating changes in capacitance and pH of a media according to time when various bias voltages are applied to a cell culture chip according to an embodiment of the present invention;

FIG. 6A is a graph illustrating a correlation between capacitance and pH of a media according to time when a specific bias voltage is applied to a cell culture chip according to an embodiment of the present invention;

FIG. 6B is a graph illustrating results of measuring capacitances in similar conditions as those shown in FIG. 6A, while using conventional methods; and

FIG. 7 is a graph illustrating changes in conductance and pH of a media according to time when a specific bias voltage is applied to a cell culture chip according to an embodiment of the present invention.

### DETAILED DESCRIPTION OF THE INVENTION

The present invention is described more fully hereinafter with reference to the accompanying drawings, in which exemplary embodiments of the invention are shown. The present invention may, however, be embodied in many different forms and should not be construed as limited to the exemplary embodiments set forth herein. Rather, these exemplary embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “lower” other elements or features would then be oriented “above” or “upper” relative to the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, the present invention will be described in more detail with reference to the attached drawings.

An exemplary embodiment of the present invention relates to a cell culture chip for real-time monitoring not only the states of cells attached to walls of a culture chamber, but also the states of cells floating in a culture media, in micro scales.

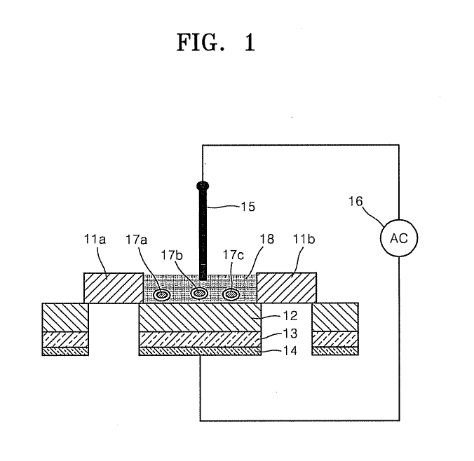

FIG. 1 is a side cross-sectional view of a cell culture chip according to an embodiment of the present invention.

Referring to FIG. 1, the cell culture chip comprises a cell culture chamber 18 formed by side walls of a non-conductive material 11a and 11b and a bottom layer of an insulating material 12 and capable of accommodating a cell culture media; a semiconductor layer 13 disposed under the bottom layer 12; a metal layer 14 disposed under the semiconductor layer 13; and an electrode 15 disposed in the cell culture chamber 18. Cells to be cultured, e.g., cells 17a-17c, are deposited into the cell media as described further herein.

The side walls 11a and 11b may be formed from any non-conductive material that may accommodate a cell culture media (e.g., cell culture media containing cells 17a-17c). For example, the non-conductive material may include, but is not limited to, one of silicone, glass, quartz, and plastics.

The bottom 12 may be composed of any insulating layer that may accommodate a cell culture media and on which cells may grow. For example, the insulating layer may be formed from a material, such as, but not limited to, SiO₂, silicone, glass, quartz, and plastics.

The semiconductor layer 13 may be a p-type or n-type semiconductor layer, and is preferably a p-type semiconductor layer. The p-type semiconductor may be a tetravalent element, such as, but not limited to, Si or Ge, doped with a trivalent element, such as, for example, B, Ga or In.

The metal layer 14 may be formed from a material, such as, but not limited to, aluminum, platinum, gold, copper, palladium, and titanium.

The electrode 15 may be formed from a material, such as, but not limited to, platinum, gold, copper, palladium, and titanium.

The metal layer 14 and the electrode 15 may be connected to a measuring means (not shown) for measuring an electrical parameter.

The electrical parameter to be measured may include one or more of: capacitance, conductance, impedance, resistance, voltage, and current.

An alternating current (AC) voltage and a direct current (DC) bias voltage may be applied between the metal layer 14 and the electrode 15. Referring to FIG. 1, an AC voltage power supply 16 is disposed between the metal layer 14 and the electrode 15. Any suitable frequency and amplitude of the AC voltage and intensity of the DC bias voltage may be applied. For example, an impedance analyzer (Solartron Analytical®, UK) may be used as the measuring means, and an AC voltage of 100 Hz, 100 mV and a DC bias voltage from −1 to 1 V may be applied between the metal layer 14 and the electrode 15.

In an embodiment of the present invention, a plurality of cell culture chambers 18 may be arranged to form a microarray and each of the cell culture chambers 18 may include the semiconductor layer 13, the metal layer 14, and the electrode 15.

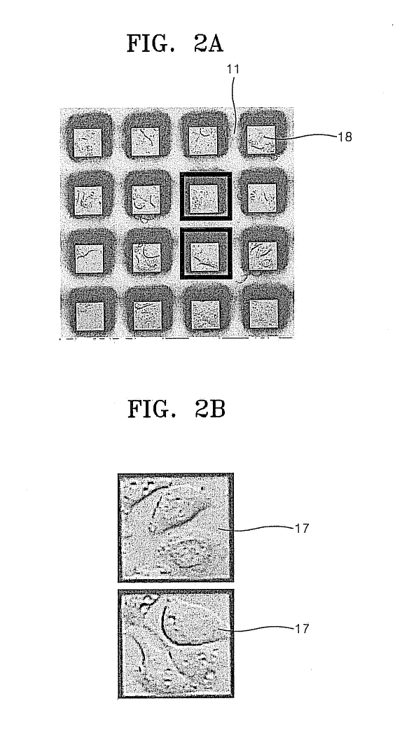

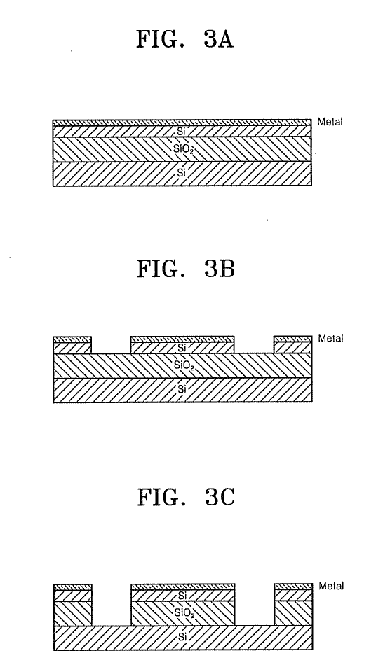

FIG. 2A is a photograph illustrating a top of a cell culture chip according to an embodiment of the present invention and FIG. 2B is an enlarged photograph illustrating rectangular portions of the cell culture chip photograph depicted in FIG. 2A.

Referring to FIG. 2A, a plurality of cell culture chambers 18 are arranged in the form of a microarray. Side walls 11 are present between the cell culture chambers 18. A bottom layer of an insulating material, a semiconductor layer, and a metal layer (not shown) are disposed in sequence below each of the cell culture chambers 18, and an electrode (not shown) is disposed in the cell culture chamber 18. The bottom layer of insulating material, the semiconductor layer, and the metal layer disposed below a cell culture chamber are separated from those layers disposed below other cell culture chambers, as illustrated in FIG. 1.

As the cells (e.g., cells 17a-17c) proliferate during the cell culture, an amount of anions in the cell culture media increases, and thus, the pH of the whole media decreases. As such, when an AC voltage is applied between the metal layer 14 and the electrode 15, the insulating layer 12 functions as a capacitor. That is, the anions become crowded on a surface of the insulating layer 12 and holes in the p-type silicone layer 13 become crowded on a surface of the insulating layer 12.

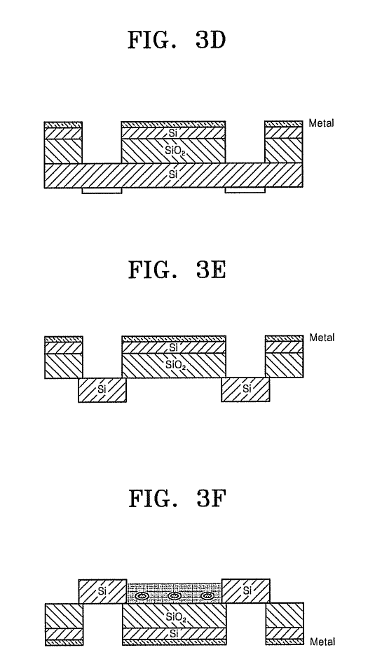

FIGS. 3A-3F schematically illustrate a method of preparing a cell culture chip according to an exemplary embodiment of the present invention.

Referring to FIG. 3, a substrate comprising, e.g., an Si layer, an SiO₂ layer, a p-type Si layer, and a metal layer are prepared in sequence as shown, e.g., in FIG. 3(a). Subsequently, the metal layer and the p-type Si layer are etched, for example, using a lithographic method as shown in FIG. 3(b), and then the SiO₂ layer is etched as shown in FIG. 3(c). Then, masks are arranged on a bottom surface of the Si layer as shown, e.g., in FIG. 3(d), and the Si layer is etched, as shown, e.g., in FIG. 3(e) to obtain the cell culture chip as shown in FIG. 3(f).

Thus configured, the structure depicted in FIG. 3(f) represents a completed cell culture chip (minus the electrode and power supply. The cell culture chip may be used for monitoring not only the states of cells attached to walls of a culture chamber, but also the states of cells floating in a culture media, in micro scales and in real time, as described below.

In an exemplary embodiment, a method of real-time monitoring a cell culture includes: placing a cell culture media and cells to be cultured, into a cell culture chamber of the cell culture chip described above in FIGS. 1 and 3A-3F; culturing the cells in the cell culture chamber; and measuring an electrical parameter between the metal layer and the electrode.

As described above, upon placing the cell culture media and cells to be cultured into the cell culture chamber, the cells (e.g., cells 17a-17c) proliferate during the cell culture, an amount of anions in the cell culture media increases, and thus, the pH of the whole media decreases. As such, when an AC voltage is applied between the metal layer 14 and the electrode 15, the insulating layer 12 functions as a capacitor. The cell culture media and the cells (e.g., cells 17a-17c) may be provided in each of desired cell culture chambers 18 using a supplying apparatus, such as a spotting apparatus.

The cells may be cultured using conventional methods. Culture conditions, such as, but not limited to, temperature, humidity, and a composition of a media, may easily be selected according to the type of cells and a purpose of culturing by a person having ordinary skill in the art.

The electrical parameter measured may include, e.g., one or more of: capacitance, conductance, impedance, resistance, voltage, and current. The electrical parameter may be measured using conventional measuring means. For example, an AC voltage and a DC bias voltage may be applied between the metal layer 14 and the electrode 15 of the cell culture chip and the related electrical parameter may be measured. Any suitable frequency and amplitude of the AC voltage and intensity of the DC bias voltage may be applied. For example, an impedance analyzer (Solartron Analytical®), UK) may be used as the measuring means, and an AC voltage of 100 Hz, 100 mV and a DC bias voltage from −1 to 1 V may be applied between the metal layer 14 and the electrode 15.

In an embodiment of the present invention, the culturing of the cells and the measuring of the electrical parameter may be simultaneously performed. That is, the electrical parameter may be measured in real time while culturing the cells.

The method according to an embodiment of the present invention may optionally include converting the measured electrical parameters into other electrical parameters.

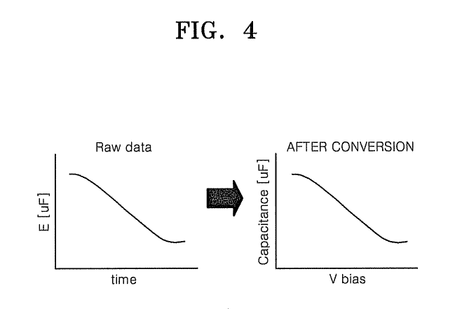

FIG. 4 schematically illustrates a process of converting electrical parameters measured between a metal layer and an electrode of a cell culture chip into other electrical parameters according to an embodiment of the present invention.

Referring to FIG. 4, epsilon (E) (unit: F) vs. time is measured using a measuring means, time is converted into voltage using voltage sweep rate, and then E is converted into capacitance by dividing the E by an area of the electrode.

The method of according to an embodiment of the present invention may optionally include converting the measured electrical parameter into a property parameter of the media.

In an embodiment of the present invention, the property parameter of the media may include, e.g., one or more of: pH, O₂ concentration, CO₂ concentration, NO concentration, and temperature.

The correlation between the electrical parameter and the property parameter of the media may be established by performing repeated experiments.

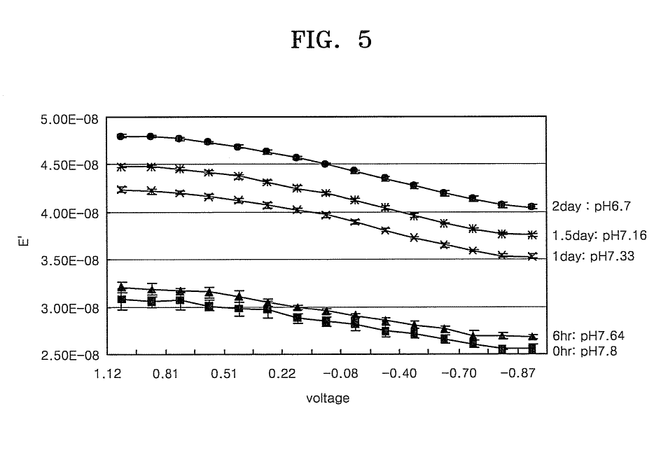

FIG. 5 is a graph illustrating changes in capacitance and pH of a media according to time when various bias voltages are applied to a cell culture chip according to an embodiment of the present invention.

Referring to FIG. 5, the capacitance is used as an electrical parameter and the pH is used as a property parameter of the media. As the time of the cell culture elapses, the pH of the media decreases and the capacitance increases.

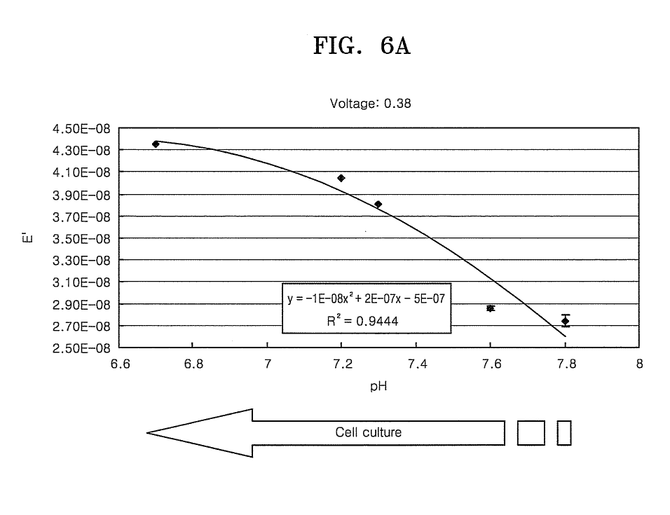

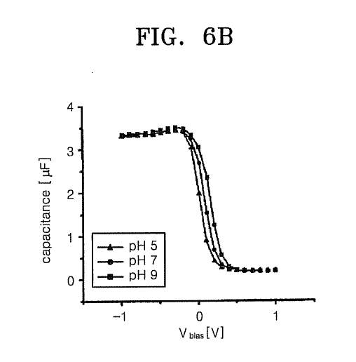

FIG. 6A is a graph illustrating a correlation between capacitance and pH of a media according to time when a specific bias voltage is applied to a cell culture chip according to an embodiment of the present invention. FIG. 6B is a graph illustrating results of measuring capacitances under similar conditions as those shown in FIG. 6A, while using conventional methods. As shown in the graph of FIG. 6B, the results are contrary to those depicted in the graph of FIG. 6A.

Referring to FIG. 6A, when a DC bias voltage of 0.38 V is applied, as the time of the cell culture elapses (in a direction from right to left of the graph), the pH decreases and the capacitance increases.

In the graph of FIG. 6A, the relationship of pH (x) with capacitance (y) may be represented by the following equation 1. The correlation R² is 0.9444, which is very high.

Equation 1
y=−1×10⁻⁸x²+2×10⁻⁷x−5×10⁻⁷ (1) (1)

For example, in the monitoring method according to an embodiment of the present invention, the capacitance as an electrical parameter may be measured and then, converted into the pH which is a property parameter of the media by using equation 1. When using the capacitance as above, the states of cells attached to a bottom of a culture chamber may be monitored.

Referring to FIG. 6B, the conventional results of measuring capacitances of solutions having different pHs using porous silicone as a substrate material of a potential difference biosensor (Meas. Sci. Technol. 7, 26-29, 1996) are contrary to the results in FIG. 6A.

It is assumed that a charge change of the media had more effect on the results of measurement in FIG. 6B than a charge change of a surface of the insulating layer.

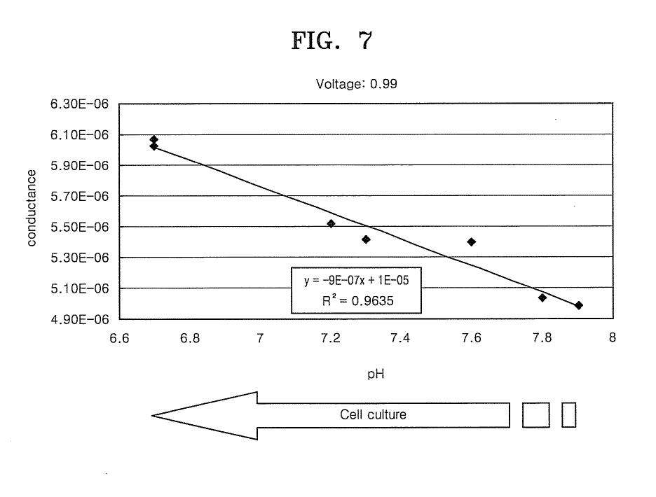

FIG. 7 is a graph illustrating changes in conductance and pH of a media according to time when a specific bias voltage is applied to a cell culture chip according to an embodiment of the present invention. The conductance indicates a change of an amount of ions in the media.

Referring to FIG. 7, when a DC bias voltage of 0.99 V is applied, as the time of the cell culture elapses (in a direction from right to left of the graph), the pH decreases and the conductance increases.

In the graph, the relationship of pH (x) with conductance (y) may be represented by the following equation 2. The correlation R² is 0.9635, which is very high.

Equation 2
y=−9×10⁻⁷x+1×10⁻⁵ (2) (2)

For example, in the monitoring method according to an embodiment of the present invention, the conductance as an electrical parameter may be measured and then, converted into the pH which is a property parameter of the media by using equation 2. When using the conductance as above, the states of cells floating in the cell chamber may be monitored.

Hereinafter, the present invention will be described in more detail with reference to the following examples. It will be understood, however, that these examples are provided for the purpose of illustration and are not intended to limit the scope of the embodiments of the invention.

In a first example, a cell culture chip was manufactured using the procedures illustrated in FIGS. 3A-3F.

The cell culture chip formed an array in which a plurality of cell culture chambers are arranged as illustrated in FIG. 2A. Side walls and a bottom defining each of the cell culture chambers were formed from Si and SiO₂, respectively. A semiconductor layer was formed from p-type Si and a metal layer was made of Al. An electrode made of platinum was used.

The cell culture chamber was formed having dimensions of 25×25×100 (μm) (width×length×height). A width of side wall of the cell culture chamber, i.e., an interval between adjacent cell culture chambers, measured 50 μm.

In a second example, it was confirmed whether a cell culture using a cell culture chip manufactured according to the procedures described in the first example could be cultured in the cell culture chambers having a bottom made of SiO₂.

DMEM media, 10% FBS and 1×antibiotics were charged into each of the cell culture chambers and HeLa cells (ATCCO Number: CCL-2™) were inoculated into the chamber at 2.5×10⁵ cells/ml. Subsequently, the cells were cultured in an incubator at 5% CO₂ concentration and 37° C. for 15 hours. FIG. 2A is a photograph illustrating a top of the cell culture chip after the cell cultured for 15 hours. FIG. 2B is an enlarged photograph illustrating rectangular portions of the cell culture chip photograph depicted in FIG. 2A.

Referring to FIGS. 2A and 2B, it can be confirmed that when the bottom of the cell culture chamber is made of SiO₂, the cell culturing can be efficiently performed.

In a third example, measurements of capacitance and pH of a media were measured according to cell culture using the cell culture chip manufactured using the procedures outlined in the first example. A correlation between the capacitance and the pH of the media was then examined.

A549s (KOREAN CELL LINE BANK, KCLB10185) were inoculated into the media at 2×10⁵ cells/ml and cultured in an incubator at 37° C. (5% CO₂ concentration, RPMI, 10% FBS, 1 xantibiotics). While an AC voltage of 100 Hz, 100 mV and a DC bias voltage from −1 to 1 V were applied between an aluminum layer and a platinum electrode in each of the cell culture chambers of the cell culture chip, the capacitance between both ends was measured using an impedance analyzer (Solartron Analytical®, UK) and the pH of the media was measured using a pH meter (Fisher Scientific®, USA). The measurements were performed at 0 hour, 6 hours, 1 day, and 2 days.

The graph depicted in FIG. 5 illustrates changes in capacitance and pH of a media according to time when various bias voltages are applied to a cell culture chip according to an embodiment of the present invention.

Referring to FIG. 5, as the cell culture time elapses, the pH of the media decreases and the capacitance increases.

FIG. 6A is a graph illustrating a correlation between capacitance and pH of a media according to time when a specific bias voltage is applied to a cell culture chip according to an embodiment of the present invention.

Referring to FIG. 6A, when a DC bias voltage of 0.38 V is applied, as the time of the cell culture elapses (in a direction from right to left of the graph), the pH decreases and the capacitance increases.

In the graph, the relationship of pH (x) with capacitance (y) may be represented by the following equation 1. The correlation R² is 0.9444, which is very high.

Equation 1
y=−1×10⁻⁸x²+2×10⁻⁷x−5×10⁻⁷ (1)

In a fourth example, conductance and pH of a media were measured according to a cell culture using the same manner as that described in the third example, and the correlation between the conductance and pH was examined.

FIG. 7 is a graph illustrating changes in conductance and pH of a media according to time when a specific bias voltage is applied to a cell culture chip according to an embodiment of the present invention.

Referring to FIG. 7, when a DC bias voltage of 0.99 V is applied, as the time of the cell culture elapses (in a direction from right to left of the graph), the pH decreases and the conductance increases.

In the graph shown in FIG. 7, the relationship of pH (x) with conductance (y) may be represented by the following equation 2. The correlation R² is 0.9635, which is very high.

Equation 2
y=9×10⁻⁷x+1×10⁻⁵ (2)

As described above, according to the present invention, not only the states of cells attached to walls of a culture chamber, but also the states of cells floating in a culture media may be monitored. The states of cells in micro scales present in a specific local position may be monitored and the cells may be monitored in real time.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A cell culture chip capable for monitoring a cell culture in real time, comprising:
a cell culture chamber formed by side walls of a non-conductive material and a bottom layer of an insulating material and capable of accommodating a cell culture media;
a semiconductor layer disposed under the bottom layer;
a metal layer disposed under the semiconductor layer; and
an electrode disposed in the cell culture chamber;
wherein the cell culture chip monitors both the states of cells attached to walls of the cell culture chamber and the states of cells floating in the cell culture media.

2. The cell culture chip of claim 1, wherein the non-conductive material is selected from the group consisting of silicone, glass, quartz, and plastics.

3. The cell culture chip of claim 1, wherein the insulating material is selected from the group consisting of SiO₂, silicone, glass, quartz, and plastics.

4. The cell culture chip of claim 1, wherein the semiconductor layer is a p-type semiconductor layer.

5. The cell culture chip of claim 1, wherein the metal layer is made of a material selected from the group consisting of aluminum, platinum, gold, copper, palladium, and titanium.

6. The cell culture chip of claim 1, wherein the electrode is made of a material selected from the group consisting of platinum, gold, copper, palladium, and titanium.

7. The cell culture chip of claim 1, wherein the metal layer and the electrode are connected to a measuring means for measuring an electrical parameter.

8. The cell culture chip of claim 7, wherein the electrical parameter is selected from the group consisting of capacitance, conductance, impedance, resistance, voltage, and current.

9. The cell culture chip of claim 1, wherein a plurality of cell culture chambers are arranged in the form of a microarray and each of the cell culture chambers includes the semiconductor layer, the metal layer, and the electrode.

10. A method of monitoring a cell culture in real time, comprising:
placing a cell culture media and cells to be cultured, into a cell culture chamber of a cell culture chip, the cell culture chip comprised of:
the cell culture chamber formed by side walls of a non-conductive material and a bottom layer of an insulating material and capable of accommodating the cell culture media;
a semiconductor layer disposed under the bottom layer;
a metal layer disposed under the semiconductor layer; and
an electrode disposed in the cell culture chamber;
culturing the cells in the cell culture chamber; and
measuring an electrical parameter between the metal layer and the electrode.

11. The method of claim 10, wherein culturing the cells and measuring the electrical parameter are simultaneously performed.

12. The method of claim 10, wherein the electrical parameter is selected from the group consisting of capacitance, conductance, impedance, resistance, voltage, and current.

13. The method of claim 10, further comprising converting the measured electrical parameter into a property parameter of the media.

14. The method of claim 13, wherein the property parameter is selected from the group consisting of pH, O₂ concentration, CO₂ concentration, NO concentration, and temperature.
